(19) United States
(12) Patent Application Publication
(10) Pub. No.: US 2022/0135413 A1
(43) Pub. Date: May 5, 2022

(54) Synthesis of Ammonia Using Cycle-Generated Hydrogen Sulfide

(71) Applicant: Wisconsin Alumni Research Foundation, Madison, WI (US)

(72) Inventors: Robert Hamers, Madison, WI (US); Benjamin Bachman, Madison, WI (US)

(21) Appl. No.: 17/514,956

(22) Filed: Oct. 29, 2021

Related U.S. Application Data
(60) Provisional application No. 63/107,610, filed on Oct. 30, 2020.

(51) Int. Cl. C01C 1/02; C01B 17/74; B01J 19/24; C07C 273/10

(52) U.S. Cl. CPC C01C 1/026; C01B 17/74; C07C 273/10; B01J 19/245; B01J 19/2445; B01J 19/2465

(57) ABSTRACT

Improved methods of synthesizing ammonia from hydrogen sulfide and lithium nitrate are disclosed. Specifically, in a continuous cycle, hydrogen sulfide reactant is regenerated from the elemental sulfur that is extracted from a product of the ammonia synthesis, and the regenerated hydrogen sulfide is fed back into the ammonia synthesis reaction. The cycle that regenerates the hydrogen sulfide uses either a water-containing or a water and carbon-containing feedstock to facilitate the regeneration of the hydrogen sulfide from the elemental sulfur.

### CROSS REFERENCE TO RELATED APPLICATIONS

This application claims the benefit of U.S. provisional Application No. 63/107,610, filed Oct. 30, 2020, which is incorporated by reference herein in its entirety.

### STATEMENT REGARDING FEDERALLY SPONSORED RESEARCH OR DEVELOPMENT

This invention was made with government support under 1839174 and 1503408 awarded by the National Science Foundation. The government has certain rights in the invention.

### FIELD OF THE INVENTION

This disclosure is directed to methods of producing ammonia using a continuously operating chemical cycle.

### BACKGROUND OF THE INVENTION

There is an ever-increasing demand for ammonia (NH₃) based fertilizers, as such fertilizers are necessary to feed the growing human population. At present, the only method used to produce NH₃ at commercial scale is the Haber-Bosch process, an energy and resource intensive process for directly combining elemental hydrogen and nitrogen. The energy requirements of the Haber-Bosch process and the seemingly ever-increasing demand for NH₃ have motivated the development of alternative, industrial-scale processes. However, to displace the existing commercial process, any alternative will need to use less energy, ideally from a renewable source, while being able to operate continuously.

U.S. Pat. No. 10,221,075 to Benjamin Bachman, a co-inventor of this application, discloses a chemical cycle for producing ammonia from nitrogen and hydrogen sulfide-containing feedstocks (the Bachman cycle). Within the Bachman cycle, lithium nitride is reacted with hydrogen sulfide, which is added to the cycle from an external source, to directly form ammonia and lithium sulfide. The ammonia product is then removed from the cycle. Metallic lithium is extracted from the resulting lithium sulfide (along with elemental sulfur, which is also removed from the cycle). The metallic lithium can then be reacted with the nitrogen that is added to the cycle to form lithium nitride, thus completing the cycle.

The Bachman cycle is more energy efficient than the both the Haber-Bosch process and the cycle proposed by J. McEnaney et al. (Energy Environ. Sci., 2017, 10, 1621-1630), which uses water instead of hydrogen sulfide.

However, the Bachman cycle requires the continuous addition of hydrogen sulfide reactant into the cycle, and the corresponding removal of unneeded elemental sulfur, which is a byproduct of the extraction of the metallic lithium from the lithium sulfide produced in the ammonia synthesis step. Accordingly, there is a need for methods that improve the Bachman cycle by regenerating the needed hydrogen sulfide from the elemental sulfur byproduct.

### BRIEF SUMMARY

We disclose herein an improved Bachman cycle that includes a second chemical loop, where the hydrogen sulfide reactant is regenerated from the elemental sulfur byproduct. The second chemical loop forms part of the larger continuous chemical cycle, and can be configured or combined with the original Bachman cycle in ways that minimize or effectively remove excess carbon dioxide, oxygen, and/or other unwanted byproducts from the system, or that use any excess carbon dioxide to make other useful products.

Accordingly, this disclosure encompasses a method for making ammonia that includes the steps of (a) reacting lithium nitride with hydrogen sulfide to produce lithium sulfide and ammonia; (b) reacting lithium with nitrogen to produce the lithium nitride that is used as a reactant in step (a); (c) extracting lithium from the lithium sulfide that is produced in step (a) to produce the lithium that is used as a reactant in step (b), whereby a sulfur byproduct is produced; and (d) reacting the sulfur byproduct of step (c) with a feedstock that includes water, hydrogen, a carbon-containing composition, or both water and a carbon-containing compound, to produce the hydrogen sulfide that is used as a reactant in step (a).

In some embodiments, steps (a)-(d) occur simultaneously.

In some embodiments, steps (a)-(d) occur within one or more interconnected reaction chambers.

In some embodiments, steps (a)-(c) occur in a reaction chamber in a flux of molten salts.

In some embodiments, steps (a)-(d) occur in a multi-loop chemical cycle. In some such embodiments, the chemical cycle operates continuously.

In some embodiments, the nitrogen used as a reactant in step (a) is added to the interconnected reaction chambers or to the chemical cycle from a source that is external to the interconnected reaction chambers or to the chemical cycle. In some such embodiments, the nitrogen is added simultaneously with the occurrence of steps (a)-(d).

In some embodiments, the nitrogen is added continuously as steps (a)-(d) occur.

In some embodiments, the feedstock used as a reactant in step (d) is added to the interconnected reaction chambers or to the chemical cycle from a source that is external to the interconnected reaction chambers or to the chemical cycle. In some such embodiments, the feedstock is added simultaneously with the occurrence of steps (a)-(d).

In some embodiments, the feedstock is added continuously as steps (a)-(d) occur.

In some embodiments, the ammonia produced as a product in step (a) is removed from the interconnected reaction chambers or from the chemical cycle. In some such embodiments, the ammonia is removed simultaneously with the occurrence of steps (a)-(d).

In some embodiments, the ammonia is removed continuously as steps (a)-(d) occur.

In some embodiments, one or more of steps (a)-(d) occur at less than 2.0 atmospheres of pressure. In some such embodiments, one or more of steps (a)-(d) occur at less than 1.5 atmospheres of pressure. In some such embodiments, one or more of steps (a)-(d) occur at atmospheric pressure.

In some embodiments, the feedstock includes water, but does not include a carbon-containing composition. In some such embodiments, the method does not generate carbon dioxide.

In some embodiments, step (d) is performed using the Reverse Claus Process. In some such embodiments, the Reverse Claus Process is performed at a temperature above 300° C.

In some embodiments, step (d) produces a sulfuric acid byproduct. In some such embodiments, performing step (d) further includes removing the sulfuric acid byproduct from the one or more reaction chambers or from the chemical cycle. In some such embodiments, the sulfuric acid byproduct is removed to a separate reaction chamber.

Some embodiments further include the step of decomposing the sulfuric acid byproduct. In some such embodiments, the sulfuric acid byproduct is decomposed by thermal decomposition.

In some embodiments, step (d) directly or indirectly produces one or more undesirable byproducts, including without limitation water, sulfur dioxide, sulfur trioxide, or other heavy gases. In some such embodiments, step (d) further includes separating the hydrogen sulfide from the one or more undesirable byproducts. In some embodiments, the hydrogen sulfide is separated from the one or more undesirable byproducts by cooling or by compression.

In some embodiments, step (d) directly or indirectly produces oxygen as a byproduct. In some such embodiments, the oxygen byproduct is removed from the one or more reaction chambers or from the chemical cycle.

In some embodiments, the feedstock includes both water and a carbon-containing composition. In some such embodiments, the carbon-containing composition may include one or more of biomass, a hydrocarbon, a plastic, or an oxide of carbon. In some such embodiments, the carbon-containing composition may include methane, biomass, plastic, or combinations thereof.

Non-limiting examples of biomass that could be used in the feedstock include agricultural residues, wood and wood derivatives, municipal waste, an energy crop, residual solids from industrial processes, algae and algae derivatives, or mixtures thereof.

In some embodiments, the biomass includes one or more of cellulose, hemicellulose, lignin, or other carbohydrates.

In some embodiments, the plastic used in the feedstock includes an organic polymer.

In some embodiments, the plastic is derived from waste.

In some embodiments, the plastic includes a polyamide, a polycarbonate, a polyester, a polyethylene, a polypropylene, a polystyrene, a polyurethane, a polyvinyl chloride, a polyvinylidene chloride, an acrylonitrile butadiene styrene, or mixtures thereof.

In some embodiments, the feedstock further includes one or more contaminants.

In some embodiments, the mole ratio of C:O in the water and carbon-containing composition that are together included in the feedstock is from 0.8:2 to 1.2:2. In some such embodiments, the mole ratio of C:O in the water and carbon-containing composition together is from 0.9:2 to 1.1:2. In some such embodiments, the mole ratio of C:O in the water and carbon-containing composition together is from 0.95:2 to 1.05:2. In some such embodiments, the mole ratio of C:O in the water and carbon-containing composition together is from 0.99:2 to 1.01:2. Ideally, the mole ratio of C:O in the water and carbon-containing composition together is 1:2.

In some embodiments, step (d) produces a carbon dioxide byproduct. In some such embodiments, the method further includes reacting the carbon dioxide byproduct with the ammonia produced in step (a) to produce urea. In some such embodiments, the step of producing urea includes pressurizing the reaction chamber where the reaction takes place.

In some embodiments, the step of producing urea occurs at a pressure above 10 bar. In some such embodiments, the step of producing urea occurs at a pressure above 100 bar.

In some embodiments, performing step (d) further includes removing the carbon dioxide byproduct from the one or more reaction chambers where it is produced or from the chemical cycle. In some such embodiments, the carbon dioxide byproduct is frozen.

In some embodiments, the carbon dioxide byproduct is sequestered.

In some embodiments where the carbon dioxide byproduct is removed, the carbon dioxide is reacted with ammonia to produce urea. In some such embodiments, the reaction of the carbon dioxide byproduct with ammonia occurs in a separate reaction chamber from the reaction chamber where the ammonia of step (a) is produced.

Further objects, features and advantages of the invention will be apparent from the following detailed description when taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The disclosure will be better understood and features, aspects and advantages other than those set forth above will become apparent when consideration is given to the following detailed description. Such detailed description makes reference to the following drawings.

FIG. 1 is a simplified reaction diagram of the proposed reaction cycle compared to the cycle proposed by McEnaney.

FIG. 2 is a diagram illustrating the hypothetical industrial implementation of the proposed process. 1) Reaction between Lithium and Nitrogen to form Lithium Nitride. 2) Reaction between Lithium Nitride and Hydrogen Sulfide to form Ammonia and Lithium sulfide, the ammonia formed here is separated by density and compressed for storage. 3) Electrolysis of molten lithium sulfide to form gaseous sulfur and molten lithium. 4) Reaction between sulfur and water vapor to form hydrogen sulfide, sulfur dioxide, and sulfuric acid. 5) Decomposition of sulfuric acid into sulfur trioxide and water, then into sulfur dioxide and oxygen in a bayonet reactor over catalysts. All gasses besides oxygen are scrubbed from the solution by passing though water before being re-introduced to reactor 4. Lime is used to capture any residual sulfur compounds before venting oxygen. 6) Separation of hydrogen sulfide by cooling. Condensed water and the sulfur dioxide dissolved in it are re-introduced to chamber 4.

While the invention is susceptible to various modifications and alternative forms, specific embodiments have been shown by way of example in the drawings and are herein described in detail. The description of specific embodiments is not intended to limit the invention to the particular forms disclosed, but on the contrary, is intended to cover all modifications, equivalents, and alternatives falling within the spirit and scope of the invention as defined by the appended claims.

### DETAILED DESCRIPTION

### I. In General

This invention is not limited to the particular methodology, protocols, materials, and reagents described, as these may vary. It is also to be understood that the terminology used in this disclosure is for the purpose of describing particular embodiments only and is not intended to limit the scope of the present invention, which will be limited only by the language of the appended claims.

As used in this disclosure and in the appended claims, the singular forms “a”, “an”, and “the” include plural reference unless the context clearly dictates otherwise. The terms “a” (or “an”), “one or more” and “at least one” can be used interchangeably. The terms “comprising”, “including”, and “having” can also be used interchangeably.

Unless defined otherwise, all technical and scientific terms used in this disclosure, including element symbols, have the same meanings as commonly understood by one of ordinary skill in the art. Chemical compound names that are commonly used and recognized in the art are used interchangeably with the equivalent IUPAC names.

All publications and patents specifically mentioned in this disclosure are incorporated by reference for all purposes.

### II. The Invention

The original Bachman cycle (disclosed in U.S. Pat. No. 10,221,075) is a method for making ammonia that includes the steps of (a) reacting lithium nitride with hydrogen sulfide to produce lithium sulfide and ammonia; (b) reacting lithium with nitrogen to produce the lithium nitride that is used as a reactant in step (a); and (c) extracting lithium from the lithium sulfide that is produced in step (a) to produce the lithium that is used as a reactant in step (b), which also produces a sulfur byproduct.

In this application, we disclose an improved Bachman cycle that includes a second loop adding the step of (d) reacting the sulfur byproduct of step (c) with a feedstock that includes water, a carbon-containing composition, or both, to produce the hydrogen sulfide that is used as a reactant in step (a). The second loop is incorporated into the original Bachman cycle to create a unified chemical cycle to which nitrogen and the feedstock can be continuously added, and from which the ammonia product can be continuously removed.

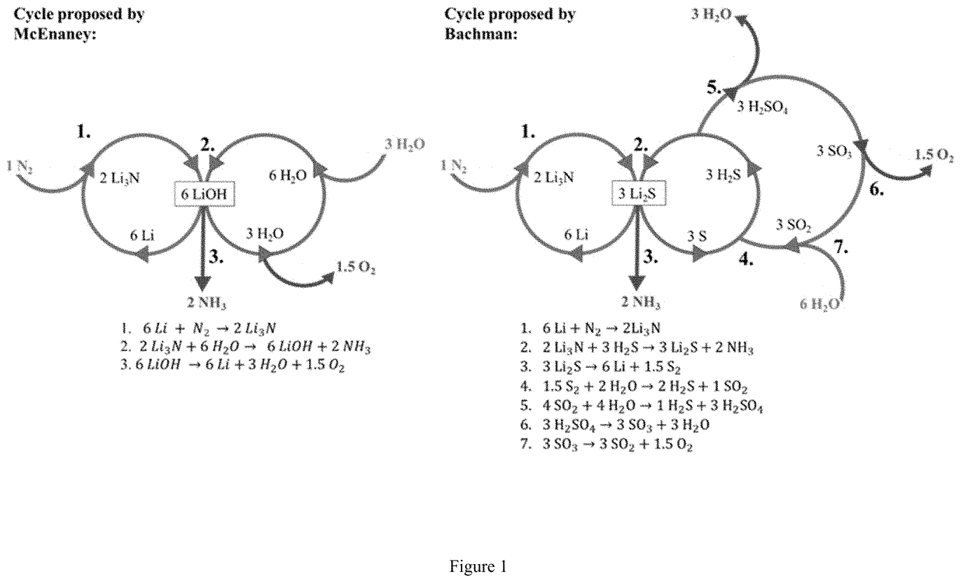

When the feedstock includes water without a carbon-containing composition, the Reverse Claus Process may be used within the second loop to regenerate the hydrogen sulfide (see FIG. 1, right panel). The oxygen byproduct can also be continuously removed from the system.

When the feedstock includes both water and a carbon-containing composition (such as biomass or plastic), the regeneration within the second loop produces carbon dioxide, which can also be removed from the system and converted into other useful products, such as urea. The C:O mole ratio may be carefully adjusted to minimize the production of other unwanted byproducts.

Biomass that can be Used in the Feedstock for Regenerating Hydrogen Sulfide from Sulfur

As used in this disclosure and in the appended claims the term “biomass” refers to, without limitation, organic materials produced by plants (e.g., wood, leaves, roots, seeds, stalks, etc.), and microbial and animal metabolic wastes. Common biomass sources include: (1) agricultural residues, such as corn stalks, straw, seed hulls, sugarcane leavings, bagasse, nutshells, and manure from cattle, poultry, and hogs; (2) wood materials, such as wood or bark, sawdust, timber slash, and mill scrap; (3) municipal waste, such as waste paper and yard clippings; (4) energy crops, such as poplars, willows, switch grass, pine, miscanthus, sorghum, alfalfa, prairie bluestream, corn, soybean, and the like; (5) residual solids from industrial processes, such as lignin from pulping processes, acid hydrolysis, or enzymatic hydrolysis; and (6) algae-derived biomass, including carbohydrates and lipids from microalgae (e.g., Botyococcus braunii, Chlorella, Dunaliell tertiolecta, Gracilaria, Pleurochyrsis carterae, and Sargassum) and macroalgae (e.g., seaweed).

The term “biomass” also refers more broadly to the primary building blocks of the above, namely, lignin, cellulose, hemicellulose, and derivatives thereof, along with other carbohydrates, such as saccharides of any size (i.e. monosaccharides, disaccharides, trisaccharides, oligosaccharides, or polysaccharides), sugars, and starches, among others. A person of ordinary skill in the art will appreciate that different terms can be used to refer to the same molecules depending on the context. For example, the term “sugar” can include simple sugars, e.g. monosaccharides like glucose or fructose, or complex sugars, e.g. disaccharides like sucrose or maltose or even larger molecules.

The following examples are offered for illustrative purposes only and are not intended to limit the scope of the invention in any way. Indeed, various modifications of the invention in addition to those shown and described herein will become apparent to those skilled in the art from the foregoing description and the following examples, falling within the scope of the appended claims.

### III. Examples

### Example 1: Organic-Hydrocarbon-Sulfur-Ammonia (OHSA) Cycle

### Background

The adoption of the Haber-Bosch process and the resulting surplus of ammonia (NH₃)-based fertilizer in 1913 allowed for the massive population explosion over the last century. [1] This process operates through the direct combination of elemental nitrogen sequestered from ambient air with hydrogen obtained from steam-reforming methane to produce ammonia. While the Haber-Bosch process has been an undoubted success, our reliance upon it has not come without cost; due to the need for high temperatures and pressures (˜426° C. and ˜200 atm), this process uses roughly 1% of all energy generated on Earth. [2] Further, the steam reforming of methane to generate hydrogen for this process is responsible for ˜0.93% of all carbon dioxide emissions on Earth. [3]

With anthropomorphic climate change becoming more apparent, and with a limited global supply of methane, alternative methods of producing ammonia, utilizing renewable energies and alternative sources of hydrogen, have become more attractive. One such method proposed by McEnaney et al in 2017 uses water as the source of hydrogen in a cyclic process that transforms nitrogen fixated by lithium into ammonia and lithium hydroxide at atmospheric pressure. [4] In this process, the elemental lithium is recycled from the hydroxide using high-temperature electrolysis yielding ammonia at ˜14 kWh/kg NH₃. Comparing this efficiency to the conventional Haber-Bosch process which produces ammonia between 10-15 kWh/kg NH₃, this cyclic electrification strategy holds promise, and creates nothing but NH₃ and Oxygen (O₂).

However, there are downsides to this process, primarily the cost of electricity and the material considerations for the electrolysis of lithium hydroxide (LiOH). The price of electricity on average is on the order of three times more expensive than thermal energy, so a process driven primarily through electrolysis would need to be approximately three times more efficient to compete economically with the Haber-Bosch. [5] The second downside is the difficult electrolysis of LiOH, a material produced when water is used as feedstock to protonate Li₃N. Unless heated to above 934° C. to decompose to lithium oxide (Li₂O), LiOH will scavenge a hydrogen from the ammonia, decreasing the efficiency of this process. LiOH is also difficult to decompose by electrolysis as it suffers from low efficiencies (84-86%) and generates superoxide oxide ions (O²) that etch the anode, forming carbon dioxide (CO₂) if using graphite. [6]

Noting these downsides to using water, it is prudent to look towards other sources of hydrogen that might replace H₂O in this cyclic reaction scheme. A short list of possible materials that are abundant or industrially available include: Methane (CH₄), hydrogen (H₂), hydrogen chloride (HCl), hydrogen bromide (HBr), hydrogen iodide (HI), and hydrogen sulfide (H₂S).

The direct reaction between CH₄ and Li₃N was shown not to produce NH₃; this is discussed in more detail in the next section. H₂ may also be ruled out as a potential feedstock as it forms LiH and LiNH₂ at low temperatures where it is thermodynamically favorable. [6] At higher temperatures between 300-450° C., the LiNH₂ will decompose to form NH₃ and LiNH, which will further break down between 550-700° C. into Li (1), N₂ (g), and H₂ (g). Further complicating the use of H₂ is that the formation of LiNH₂ and LiH forms a protecting layer over the Li₃N, limiting further reaction and nessesitating higher pressures or ball milling to force the reaction to completion. HCl, HBr, and HI are all industrially available materials, but all react with NH₃ to form a stable NH₄X (X=Cl, Br, I) compound that requires additional energy to decompose.

Finally, we are left with H₂S, which reacts exothermically with Li₃N to form lithium sulfide (Li₂S) and NH₃ (it may also form ammonium sulfide (NH₄)₂S, but this compound breaks down at room temperature). Unlike H₂O which forms lithium hydroxide (LiOH), H₂S forms Li₂S; as Lithium hydrosulfide (LiSH) is unstable and does not exist in an anhydrous form. Li₂S is also easier to electrowon into Li metal and gaseous sulfur (S₂) as it requires a lower voltage and forms the sulfite ion (S²⁻) which, while able to etch quartz at high temperatures, has been shown not to etch magnesia crucibles or graphite anodes during molten-salt electrolysis. [7-9] Magnesia has also been shown good resistance to molten lithium, but fails at around or above 1000° C.[10] Molybdenum or MoS₂ may be the ideal crucibles for this high temperature electrolysis.

Water Versus Hydrogen Sulfide Feedstocks:

The difference in electrical energy required to run cycles using H₂O and H₂S as feedstocks are calculated using entropy and enthalpy values from the JANAF-NIST thermochemistry tables.

First, the decomposition voltages of the litigated anions (LiOH and Li₂S) at their melting point is calculated from the Gibbs free energy, starting with H₂O feedstock, which generates LiOH:

| 700K | 6 LiOH (l) | 6 Li (l) | 3 H₂O (g) | 1.5 O₂ (g) |
|---|---|---|---|---|
| $Δ H ( \frac{kJ}{mol} )$ | −486.298 | 0 | −245.632 | 0 |
| $Δ S ( \frac{J}{K} )$ | 95.101 | 59.283 | 218.739 | 231.466 |

$Δ G = 2190.89 ( \frac{kJ}{mol} ) + 788.51 ( \frac{J}{K} ) * 700 K = 1628.9 \frac{kJ}{mol}$

To calculate the theoretical voltage from the Gibbs free energy the following equation is used:

$${E}_{cell} = \frac{- Δ {G}_{rxn}}{n F} = \frac{- 1628.9 \frac{kJ}{mol} * \frac{1000 J}{1 kJ} * \frac{C * V}{J}}{6 {e}^{-} * 96485 \frac{C}{mol}} = 2.81 V {LiOH}^{*}$$

To calculate energy required to produce ammonia the following equation is used considering the number of moles of NH₃ produced per electron:

$$\frac{\# mol {e}^{-}}{\# mol {NH}_{3}} * \frac{1 F}{1 mol {e}^{-}} * \frac{96 , 485 C}{1 F} * V * \frac{1 kWh}{3 , 600 , 000 C * V} * \frac{1 mol {NH}_{3}}{17.0 \; 31 g {NH}_{3}} * \frac{1000 g}{1 kg} = X \frac{kWh}{kg {NH}_{3}}$$

$$\frac{6 mol {e}^{-}}{2 mol {NH}_{3}} * \frac{1 Farad}{1 mol {e}^{-}} * \frac{96 , 485 C}{1 F} * 2.81 V = 483 , 389 J * \frac{1 kWh}{3 , 600 , 000 J} * \frac{1 mol {NH}_{3}}{17.031 g} * \frac{1000 g}{1 kg} = 13.28 \frac{{kWh}^{**}}{kg {NH}_{3}} , 814.4 \frac{kJ}{mol}$$

The calculated voltage from McEnaney's paper is listed as “2.8 V”, but due to overpotentials the measured voltage is “˜3 V”.
*Using 3 V instead of 2.8 V gives a value of ˜14.2 kWh/kg NH₃

To calculate the energy required when H₂S is used as the feedstock, the following calculations are used:

| 600K* | 6 Li₂S (l)** | 12 Li (l) | 3 S₂ (g) |
|---|---|---|---|
| $Δ H ( \frac{kJ}{mol} )$ | −423.68 | 0 | 0 |
| $Δ S ( \frac{J}{K} )$ | 113.13 | 55.283 | 60.078 |

$Δ G = 2542.08 ( \frac{kJ}{mol} ) + 164.84 ( \frac{J}{K} ) * 1200 K = 2443.17 \frac{kJ}{mol}$
${E}_{cell} = \frac{- Δ {G}_{rxn}}{nF} = \frac{- 2443.17 \frac{kJ}{mol} * \frac{1000 J}{1 kJ} * \frac{C * V}{J}}{12 {e}^{-} * 96485 \frac{C}{mol}} = 2.11 V {Li}_{2} S$

As before, the energy required to produce a mole of NH₃ is calculated below using the decomposition voltage calculated above and the needed ratio of NH₃ to electrons:

$$\frac{12 mol {e}^{-}}{4 mol {NH}_{3}} * \frac{1 Farad}{1 mol {e}^{-}} * \frac{96 , 485 C}{1 F} * 2.11 V = 583 , 755 J * \frac{1 kWh}{3 , 600 , 000 J} * \frac{1 mol {NH}_{3}}{17.031 g} * \frac{1000 g}{1 kg} = 9.96 \frac{{kWh}^{** *}}{kg {NH}_{3}} , \\ 610.76 \frac{kJ}{mol}$$

The melting point of Li₂S varies by source from 900-1370° C. However, the highest temperature values of H and S were measured at 600 K, less than half the possible required temperature
*The entropy and enthalpy values listed for Li₂S are from reference and is at 600 K. Like the melting point, there is no consensus on these values.
**Note that decomposition voltages, and therefore the electrical energy requirements for electrochemical NH₃ production, decreases with increasing temperature. Li₂S electrolysis will likely be operated at 1200 K (600 K hotter), so the required voltage and electrical energy will be lower. If LiOH electrolysis was similarly run at 600 K hotter (1300 K), the decomposition voltage and energy requirement for NH₃ production would be 2.03 V and 9.61 kWh/kg NH₃, respectively, a 27% decrease in electrical energy requirement. If a similar energy reduction is shown with Li₂S, the total voltage and energy required would be 1.46 V and 6.89 kWh/kg NH₃, respectively.

From the data listed above, using H₂S as a feedstock to produce Li₂S requires significantly less electrical energy than using H₂O to create LiOH due to the lower electrical potential required for electrolysis.

Sulfur-Reforming Biomass:

The reaction between CH₄ and Li₃N at the melting point of Li₃N (813° C.) has never been demonstrated in the literature. It was expected that Reaction 1 would occur:

$$3 {CH}_{4} + 1 {Li}_{3} N \to 1.5 {Li}_{2} {C}_{2} + 1 {NH}_{3} + 4.5 {H}_{2} \\ Δ G = 181.6 \frac{kJ}{mol} ( 293 K ) , - 60.39 \frac{kJ}{mol} ( 1086 K ) \qquad Reaction 1$$

However, the products of this reaction indicate that Reaction 2 took place instead, even though at 813° C., this reaction is less exergonic:

$$3.75 {CH}_{4} + 1 {Li}_{3} N \to 0.25 {Li}_{2} {C}_{2} + 1.5 {C}_{3} {N}_{4} + 7.5 {H}_{2} \\ Δ G = 107.2 \frac{kJ}{mol} ( 293 K ) , - 46.76 \frac{kJ}{mol} ( 1086 K ) \qquad Reaction 2$$

No NH₃ was detected during the reaction, and both Li₂C₂ and C₃N₄ were measured in the sample crucible after cooling to room temperature. This unexpected result motivates the use of an intermediate compound to transfer hydrogen from hydrocarbon materials to Li₃N, similar to how hydrogen is removed from CH₄ using H₂O before reacting with Na in the Haber-Bosch process. This high-temperature, endothermic, hydrogen-extraction method is known as steam reforming; the chemical formula is detailed in Reaction 3 along with the enthalpy and entropy values used to calculate the free energy.

$$1 {CH}_{4} + 1 {H}_{2} O \to 1 {CO}_{2} + 3 {H}_{2} \\ Δ G = - 227.2 \frac{kJ}{mol} - ( 0.2542 \frac{kJ}{K} * 1200 K ) = - 83.8 k J \qquad Reaction 3$$

The carbon monoxide (CO) generated by this process is further reacted in a lower-temperature, exothermic, reaction known as the water gas shift reaction; similarly detailed in Reaction 4:

$$1 CO + 1 {H}_{2} O \to 1 {CO}_{2} + 1 {H}_{2} \\ Δ G = - 37.882 \frac{kJ}{mol} - ( - 0.03966 \frac{kJ}{K} * 800 K ) = - 19.12 kJ \qquad Reaction 4$$

As detailed previously, H₂ gas does not react favorably with Li₃N to produce NH₃. Therefore, instead of using the steam-reforming/water gas shift reactions, similar reactions using elemental sulfur will be used to generate H₂S. The first of the two reactions, hereinafter referred to as “sulfur-reforming”, is a high-temperature, exothermic, hydrogen-extraction method detailed in Reaction 5 along with the enthalpy and entropy values used to calculate the free energy:

$$3 {CH}_{4} + 6 {S}_{2} \to 3 {CS}_{2} + 6 {H}_{2} S \\ Δ G = - 301.28 \frac{kJ}{mol} - ( 0.8674 \frac{kJ}{K} * 1000 K ) = - 1 \; 145.4 kJ \qquad Reaction 5$$

The carbon disulfide (CS₂) generated by this process may be further reacted in a slightly lower or similar temperature reaction vessel with H₂O, similarly, detailed in Reaction 6:

$$3 {CS}_{2} + 6 {H}_{2} O \to 3 {CO}_{2} + 6 {H}_{2} S \\ Δ G = - 196.38 \frac{kJ}{mol} - ( 0.02492 \frac{kJ}{K} * 900 K ) = - 1 \; 74.62 kJ \qquad Reaction 6$$

Alternatively, non-methane hydrocarbons may be used as feedstock in Reaction 7. If biomass is used, represented as cellulose in reaction 7, more CO₂ is produced by the reaction given the same amount of input sulfur. The high thermodynamic stability of CO₂ coupled with the significant increase in entropy from generating more moles of gas make Reaction 7 more exergonic than Reaction 6:

$$1 {C}_{6} {H}_{10} {O}_{5} + 7 {H}_{2} O + 6 {S}_{2} \to 6 {CO}_{2} + 12 {H}_{2} S \\ Δ G = - 711.43 \frac{kJ}{mol} - ( 0.1750 \frac{kJ}{K} * 900 {K}^{*} ) = - 2286.44 kJ \qquad Reaction 7$$

*It should be noted that this reaction will likely be run at 1000 K. Reaction 7 is calculated at 900 K because the literature values for the enthalpy and entropy of microcrystalline cellulose are for 580° C.[11]

The entire reaction cycle to generate NH₃ using cellulose as feedstock as in reaction 7 is detailed in Table 1.

TABLE 1
Four-step reaction cycle compromising the Organic
Hydrocarbon Sulfur Ammonia (OHSA) cycle

| | | | Electrical | Thermal | |
|---|---|---|---|---|---|
| | | Temperature | requirement | requirement | ΔG |
| # | Reaction cycle: | (° C.) | (kJ) | (kJ) | (kJ) |
| 1 | 24 Li + 4 N₂ → 8Li₃N | 180.5 | | 180.32 | −879.8 |
| 2 | 8 Li₃N + 12 H₂S | 30 | | 0 | −3697.1 |
| | → 12 Li₂S + 8 NH₃ | | | | |
| 3 | 12 Li₂S → 24 Li + 6 S₂ | 938 (600 K) | (4886.3) * | 876.36** | 4886.3 |
| 4 | 6 S₂ + 1 C₆H₁₀O₅ + 7 H₂O | 700 (900 K) | | 491.54 | −2286.4 |
| | → 6 CO₂ + 12 H₂S | | | | |

| | | | |
|---|---|---|---|
| Net Reaction: | Total: | 610.8 kJ/mol NH₃ | −247.1 kJ/ |
| 4 N₂ + 1 C₆H₁₀O₅ + 7 H₂O | | (assuming only | mol |
| → 12 CO₂ + 8 NH₃ | | electrolysis matters) | |
| | | 9.96 kWh/kg NH₃ | −4.03 kWh/ |
| | | (13.12 with thermal, | kg NH₃ |
| | | no heat recycling) | |

* The electrical requirement is calculated for Li₂S at 600 K.
**The heat capacity of Li₂S is unknown, so the thermal requirement for reaction 3 (heating Li₂S from 30 to 938° C.) was calculated using the heat capacity of S₈ at STP. The heat of fusion was not included in this calculation.

Envisioned Process:

A carbon-containing feedstock including materials such as biomass, coke, coal, methane, or petroleum-derived products such as gasoline or plastics, may be added to a vessel and weighed. In the case of biomass, the material may be shredded and compacted into the vessel, then by determining both volume and mass, water may be added to the vessel to achieve the required 7:1 molar ratio of H₂O:hydrocarbon. The feedstock may then be added to a reaction chamber (1) and sealed before being heated and exposed to gaseous sulfur.

A heated (˜400° C.) distillation column above the reaction chamber will allow for separation of gasses while allowing sulfur to condense and flow back into the chamber without excess polymerization. This distillation column may be covered in a layer of alumina to act as a catalyst for H₂S conversion. [12] A secondary distillation column may be used to condense H₂O. The primary gasses formed will be CO₂ and H₂S, with trace amounts of CO, CSO, N₂, SO₂, O₂, H₂, CH₄, H₂O, and NH₃. The type and volume of trace gasses formed may vary with the ratio of H:C:O in the reaction chamber.

These gasses may then be cooled and cryogenically separated to remove the H₂S. In another form, these gasses may be separated through exposure to NH₃ to form (NH₄)₂S (l) with H₂S, urea (l, s) and H₂S from COS, and (NH₄)₂SO₃ from SO₂. [13] The (NH₄)₂S may then be removed while the remaining reaction products may be collected or refed into reaction chamber 1. The CO₂ may then be captured or vented. Remaining gasses may be reintroduced back into reaction chamber 1.

The H₂S or (NH₄)₂S may then be transferred either as a liquid or gas to a second reaction chamber (2) to react with Li₃N. This will form NH₃, Li₂S, and (NH₄)₂S, the latter will decompose with mild heating. The NH₃ may then be collected and purified. The Li₂S may then be removed from reaction chamber 2 and added to a third reaction chamber (3).

The third reaction chamber may be composed of a refractory metal or ceramic lined crucible and may contain a molten salt eutectic. The Li₂S may then be decomposed either through electrolysis or thermal decomposition to separate the Li and S. The gaseous sulfur generated through this decomposition may then be fed back into reaction chamber 1. The Li may then be transferred to a fourth reaction chamber (4) where it may be reacted with N₂ to form Li₃N. This Li₃N may then be transferred back to reaction chamber 2.

Alternatively, the H₂S may be fed into a molten flux containing a lithium-halogen salt. Through the application of an electric potential across this lithium-containing salt, nitrogen may be reduced into nitride ions. These nitride ions will react with H₂S bubbled through the reaction chamber to form Li₂S and NH₃. The Li₂S will then be decomposed electrochemically to release sulfur which is removed from the reaction chamber.

The viscosity of molten sulfur increases exponentially with temperature due to polymerization at the melting point, then begins to decrease after ˜200° C. until its boiling point at 444° C. At ˜400° C., the viscosity of sulfur is ˜110 cP [14]

The conversion of methane and sulfur to CS₂ and H₂S is 99.9% between 400-700° C., the remainder is primarily COS. [12, 15]

Table 2 lists the boiling point of all gasses likely to be generated in the reaction between S₂ (g) and biomass.

TABLE 2
Boiling Points of Potential Products (° C.)

| Gas | SO₂ | NH₃ | OCS | H₂S | CO₂ | CH₄ | O₂ | CO | N₂ | H₂ |
|---|---|---|---|---|---|---|---|---|---|---|
| Boiling point | −10 | −33.34 | −50.2 | −60 | −78.46 | −161.5 | −183 | −191.5 | −195.8 | −252.9 |

Water Content of the Biomass Feedstock:

Step 4 of the OHSA cycle (see Table 1) reacts elemental sulfur with cellulose and water. Cellulose is used as a chemical proxy for generic biomass material. In reality, soft wood is composed of 55% cellulose, 11% hemicellulose, 26% lignin, (the reminder is composed of oils and non-organic material) and contains between 30-200% by mass H₂O on a dry-basis. [16]

The majority of the hydrogen added to this reaction cycle will be converted into H₂S, while the carbon and oxygen will be removed either though the production of CO₂ or Urea. This means that, ideally, the C:O mole ratio needs to be maintained as close as possible to 1:2. If this ratio instead includes a greater amount of oxygen, SO₂ will be produced (SO₂ byproduct can be fed back into the reaction cycle and reduced). If this ratio instead produces a greater amount of carbon CS₂, another unwanted byproduct, will be produced. The amount of water present in the biomass (or other carbon-containing) feedstock can be tuned to optimize the C:O mole ratio.

Wood, which is a non-limiting example of biomass that could be used in the disclosed method, already contains a significant amount of water. The moisture content in wood is calculated (on a dry basis) by:

$$\frac{mass of water}{mass of dried wood} * 100 = \%$$

Accordingly, when wood is said to vary between 30-200% water, it means that it contains between 23-66.7% water by weight.

Here are the calculations for determining the ideal moisture content of the feedstock on a weight % basis:

First, if we assume that all of the wood is made of cellulose (meaning 44.4% C: 49.3% O), then the mass % of water to reach the ideal 1:2 mole ratio of C:O is:

$$1 mol {C}_{6} {H}_{10} {O}_{5} * 16 \; 2.14 g / mol = 162.14 g Cellulose$$

$$7 mol {H}_{2} O * 18.02 g / mol = 126.14 g {H}_{2} O = \text{\textasciitilde} 43.8 \% by mass {H}_{2} O$$

However, as stated earlier, wood is not just cellulose. Wood typically contains ˜50% C and 44% oxygen by mass. [17] This would correspond to a formula closer to C₂₅H₁₀₂₂, which using the same calculation as above gives:

$$1 mol {C}_{25} {H}_{1} {O}_{22} * 131 \; 0.5 = 1310.5 g “ wood ”$$

$$28 mol {H}_{2} O * 18.02 = 50 \; 4.56 g {H}_{2} O = \text{\textasciitilde} 27.8 \% by mass {H}_{2} O$$

Both of these values are within the range that natural wood can produce without drying or adding additional water.

Plastic as an Alternative Feedstock:

The carbon source used as a feedstock in the disclosed method is not limited to biomass, but may include any carbon-containing substance. A non-limiting example of another carbon source that could be used is plastic. A typical single-use water bottle is made from polyethylene, with the formula C₂H₄. Doing the calculation as before we get:

$$1 mol {C}_{2} {H}_{4} * 28.05 g / mol = 28.05 g polyethylene$$

$$4 mol {H}_{2} O * 18.02 g / moll = 72.08 g {H}_{2} O = \text{\textasciitilde} 72 \% by mass {H}_{2} O$$

This means that each mol of C₂H₄ used can produce 4 mols of NH₃ (there are 12 Hydrogens). If all 100 million tonnes of polyethylene produced a year were used in this process, we could make:

$$100 * {10}^{6} \frac{tonnes}{{C}_{2} {H}_{4}} * \frac{1 , 000 , 000 g}{1 tonne} * \frac{1 mol {C}_{2} {H}_{2}}{28.05 g} * \frac{4 mol {NH}_{3}}{1 mol {C}_{2} {H}_{2}} * \frac{17.0 \; 31 g}{1 mol {NH}_{3}} * \frac{1 tonne}{1 , 000 , 000 g} = 24 \; 2.8 * \frac{tonnes}{{NH}_{3}}$$

Since only ˜175*10⁶ tonnes of NH₃ are currently produced a year, there is ˜1.3× more plastic produced every year than needed to fulfill the world's fertilizer needs using the OHSA cycle disclosed in this example.

Using one or more plastics as a feedstock has several advantages over using biomass, such as wood. First, unlike wood, plastics generally do not contain any salt or metal, which present contamination and disposal issues. Second, the method could be used as a means of disposing of plastics, thus helping to solve a significant environmental problem.

Generating Urea and Other Methods of Removing Carbon Dioxide Byproduct:

The reaction between 1 CO₂ and 2 NH₃ to form CH₄N₂O (urea) and H₂O, takes place between 180-210° C. at 150 bar of pressure. It is feasible to pressurize the reaction chamber where the H₂S+Li₃N reaction is taking place to promote this reaction. However, the reaction between CO₂ and H₂S to form H₂ and CO takes place between 600-800° C. at 1 atm. [18] At 150 bar, it is likely that this reaction will also take place. Accordingly, the proposed single-chamber generation of Urea needs to be tested experimentally.

In light of this, we proposed three non-limiting methods of using the CO₂/H₂S gas mixture. First, it may be used in a single chamber reaction at high pressures to produce urea, as suggested above.

Second, the CO₂ and H₂S could be separated, and the CO₂ could then be collected for reacting with the purified NH₃ later to produce urea.

Third, the separated/collected CO₂ could be frozen, and subsequently released or sequestered. Credits are currently available to offset the cost of geological storage, which if applied could result in a significant cost-offset.

In sum, this example describes a second newly disclosed cycle that further improves on the original Bachman cycle, by using a feedstock containing biomass, plastic, or another carbon-containing composition and water to regenerate needed hydrogen sulfide from elemental sulfur extracted from the lithium sulfide product of the ammonia production step, The water may be added to the feedstock separately, or the case of biomass, may be naturally present. Like the cycle disclosed in Example 2, this disclosed cycle is energy efficient and can operate at atmospheric pressure. However, it avoids the difficulties and expense associated with the decomposition of sulfuric acid.

### REFERENCES CITED

1. Vaclav, S., Enriching the Earth: Fritz Haber, Carl Bosch, and the Transformation of World Food Production. The MIT Press, 2001.
2. Thornley, D. P. G. a. D. P., ENERGY AND CARBON BALANCE OF AMMONIA PRODUCTION. CPL Press, 2010.
3. McEnaney, J. M., et al., Ammonia synthesis from N₂ and H₂O using a lithium cycling electrification strategy at atmospheric pressure. Energy & Environmental Science, 2017. 10(7): p. 1621-1630.
4. Natural Gas and Electricity Center Point Energy, 2020.
5. Takeda, O., et al., Electrowinning of Lithium from LiOH in Molten Chloride. Journal of The Electrochemical Society, 2014. 161(14): p. D820-D823.
6. Zhang, J. and Y. H. Hu, Decomposition of Lithium Amide and Lithium Imide with and without Anion Promoter. Industrial & Engineering Chemistry Research, 2011. 50(13): p. 8058-8064.
7. Murakami, T., et al., Electrochemical Synthesis of Ammonia and Coproduction of Metal Sulfides from Hydrogen Sulfide and Nitrogen under Atmospheric Pressure. Journal of The Electrochemical Society, 2005. 152(6).
8. II, C. L. L. a. J. B. G., Anodic Oxidation of Sulfide Ions in Molten Lithium Fluoride. J. Electrochem. Soc., October 1994. 141.
9. al, C. H. L. e., Electrochemical Generation and Measurement of Sulfide Ion in Molten LiCl—KCl Eutectic. J. Electrochem. Soc. 1973 120
10. Ballif, D. W. J. J. L. and W. W. Y. B. E. Chou, LITHIUM LITERATURE REVIEW: LITHIUM'S PROPERTIES AND INTERACTIONS. Hanford Engineering Development Laboratory, April 1978.
11. Blokhin, A. V., et al., Thermodynamic Properties of Plant Biomass Components. Heat Capacity, Combustion Energy, and Gasification Equilibria of Cellulose. Journal of Chemical & Engineering Data, 2011. 56(9): p. 3523-3531.
12. S. R. Crane, L. C., W. I. Nissen, Hydrogen Sulfide Generation by Reaction of Natural Gas, Sulfur, and Steam. UNITED STATES DEPARTMENT OF THE INTERIOR, 1981. Bureau of Mines Report of Investigations.
13. CELLI, F. J., CHEMICAL. ENGINEERING FACTORS IN THE PREPARATION OF UREA FROM CAR BONYL SULFIDE AND ANHYDROUS AMMONIA. Dissertation, The Ohio State University, 1953.
14. Weinerth, J., Pumping Molten Sulfur: A Challenge for Pump Design, Sealing Systems and Material of Construction. Brimstone STS Limited.
15. DAVID E. SMITH, R. W. T., CARBON DISULFIDE. Kirk-Othmer Encyclopedia of Chemical Technology, 2000. 4.
16. USDA Agricultural Handbook AH-188, Chapter 1, Properties of Wood Related to Drying, 1999.
17. Pettersen, R. C., The Chemical Composition of Wood, Chapter 2 in Rowall, Roger M., ed.

The Chemistry of Solid Wood. Advances in Chemistry series 207. Washington D.C.: American Chemical Society, 1984.
18. Alderman, N. P, et al., Syn-gas from waste: the reduction of CO₂ with H₂S, React. Chem. Eng., 2019, 4, 763-771.

### Example 2: Cyclic Electrification Strategy for Ammonia Production Using Lithium and Hydrogen Sulfide by the Reverse Claus Process

### SUMMARY

In this example, we disclose a new cyclic process of for making ammonia that represents a significant improvement of the previously disclosed Bachman cycle. It uses less energy than the Haber-Bosch process, ideally can operate on all-renewable power, and is able to operate continuously. In this example, we both outline the process and provide a roadmap for future research that can be used to tune the process for its most efficient and effective implementation.

This new cycle leverages multiple known reactions and combines them in a new way to form a unified cycle capable of turning water and nitrogen gas into ammonia continuously. This cycle uses both elemental sulfur and lithium to decompose water and fixate nitrogen respectively, while only generating oxygen and NH₃, with no environmental pollutants. This cycle can be run entirely on renewable power and could be integrated into a nuclear power or geothermal station to better utilize excess heat.

The steps of the full reaction are shown below, and a simplified diagram of the cycle is shown in FIG. 1 (right panel):

6Li+N₂→2Li₃N 1.

2Li₃N+3H₂S→3Li₂S+2NH₃ 2.

3Li₂S→6Li+1.5S₂ 3.

1.5S₂+2H₂O→2H₂S+1SO₂ 4.

4SO₂+4H₂O→1H₂S+3H₂SO₄ 5.

3H₂SO₄→3SO₃+3H₂O 6.

3SO₃→3SO₂+1.5O₂ 7.

Net: N₂+6H₂O→2NH₃+3H₂O+1.5O₂

### INTRODUCTION

As detailed in example 1, there is a need for a process that will transfer hydrogen from an abundant source to elemental sulfur to produce H₂S. When carbon is added to reaction between water and sulfur, the oxygen atom from the water may be removed through the generation of CO₂. However, if the generation of CO₂ is not desired, due to its impact on the climate, there are possible methods to generate H₂S from water alone.

The reverse Claus process is one such method that could be used to produce H₂S from elemental sulfur feedstocks. This method would use water as the sole source of hydrogen to produce H₂S, could be operated with all-renewable energy sources, and produce only O₂ as a byproduct. The Claus process normally operates at low temperatures (<300° C.) through the following two reactions:

2H₂S+3O₂→2SO₂+2H₂O Reaction 1:

4H₂S+2SO₂→3S+4H₂O Reaction 2:

However, when run at higher temperatures, reaction 2 may be reversed to form H₂S and SO₂ from elemental sulfur and steam; this is known as the reverse Claus process. Also formed in the reverse Claus process is sulfuric acid (H₂SO₄), a byproduct that would need to be decomposed in order to remove the oxygen (introduced by the water) from the system and recover the sulfur. This may be achieved through the thermal decomposition of H₂SO₄ to form H₂O, SO₂, and O₂. The H₂O and SO₂ could be fed back into the cycle while the O₂ is removed. This thermal decomposition of H₂SO₄ has been demonstrated in the literature as part of the Sulfur-iodine water-splitting process. The full reaction cycle coupled to the Bachman cycle is detailed in Table 3.

TABLE 3
Proposed reaction cycle with calculated energetic requirements, at various temperatures.
Variation of ΔG from energetic requirement comes from variations in temperature.

| | | | Electrical | Thermal | ΔG at |
|---|---|---|---|---|---|
| | | Temperature | requirement | requirement | STP |
| # | | (° C.) | (kJ) | (kJ) | (kJ) |
| 1 | 6 Li + N₂ → 2Li₃N | 180.5 | | −203.3 | −258.09 |
| 2 | 2 Li₃N + 3 H₂S → 3 Li₂S + 2 NH₃ | 30 | | −991.4 | −991.4 |
| 3 | 3 Li₂S → 6 Li + 1.5 S₂ | 627 | 1221.6* | 217.8** | 1221.59 |
| 4 | 1.5 S₂ + 2 H₂O → 2 H₂S + 1 SO₂ | 300 | | 107.91 | 92.23 |
| 5 | 4 SO₂ + 4 H₂O → 1 H₂S + 3 H₂SO₄ | 300 | | 316.91 | 47.08 |
| 6 | 3 H₂SO₄ → 3 SO₃ + 3 H₂O | 338 | | 287.55 | 244.34 |
| 7 | 3 SO₃ → 3 SO₂ + 1.5 O₂ | 850 | | −4.72 | 212.15 |

| | | | |
|---|---|---|---|
| Net: | Total | 610.8 kJ/mol NH₃ | 283.95 kJ/ |
| N₂ + 3 H₂O → 2 NH₃ + 1.5 O₂ | | (assuming only | mol |
| | | electrolysis matters) | |
| | | 9.96 kWh/kg NH₃ | 4.63 kWh/ |
| | | (17.5 with thermal, | kg |
| | | no heat recycling) | |

*The electrical requirement is calculated for Li₂S at 600 K.
**The heat capacity of Li₂S is unknown, so the thermal requirement for reaction 3 (heating Li₂S from 30 to 938° C.) was calculated using the heat capacity of S₈ at STP. The heat of fusion was not included in this calculation.

As shown in Table 3, this proposed cycle has the same net chemical equation as the cycle proposed by McEnaney, meaning that both are fundamentally limited by the free energy of the reaction at STP: 380.4 kJ/mol NH₃, or 5.55 kWh/kg NH₃. In an ideal system, both of these proposed cycles could be run using the same energy, so the variance from the ideal energy requirements shown in Table 3 come from practical limitations such as the formation of monohydrides on LiOH and H₂SO₄ leading to different heating requirements.

Besides theoretically requiring slightly less energy to run the herein proposed cycle has two more distinct advantages. First, the cycle proposed by McEnaney creates LiOH, which on top of the previously mentioned difficulties relating to the electrolysis of this compound, also forms a strong monohydride which needs to be thermally decomposed. While H₂SO₄ also forms a hydride during the herein disclosed cycle, this water does not undergo a phase change from liquid to gas, which is far more energy intensive than simply heating steam.

Secondly, McEnaney et al. claim that their cycle produces NH₃ at ˜14 kWh/kg NH₃, because this is the energy required for electrolysis. Unlike thermal decomposition processes where one part of the system can transfer heat to another, the energy spent on electrolysis cannot be as easily recycled, and this step is the single greatest energy sink in both cycles. By requiring approximately the same amount of energy, but having less of it sunk into electrolysis, the cycle disclosed in this example leverages one of the greatest assets of the sulfur-iodine cycle, which is the ability to run on the waste heat of nuclear reactors, solar concentrators, or geothermal energy sources, without having to wastefully convert the heat to electricity first. This means that if thermal energy was “free” for both cycles, then the herein proposed cycle would cost ˜1.6× less than the cycle proposed by McEnaney while generating the same amount of NH₃.

Besides the inevitable engineering challenges of working with hot lithium and sulfuric acid, the herein disclosed cycle also suffers from a strong reliance on thermal heat transfer. If some of the thermal energy of this cycle is lost, for example venting steam or improper insulation, this cycle may become significantly less efficient than the Haber-Bosch process. Therefore, this cycle, like the Haber-Bosch, will need to be optimized for efficient heat transfer between steps in the reaction.

Envisioned Industrial Process:

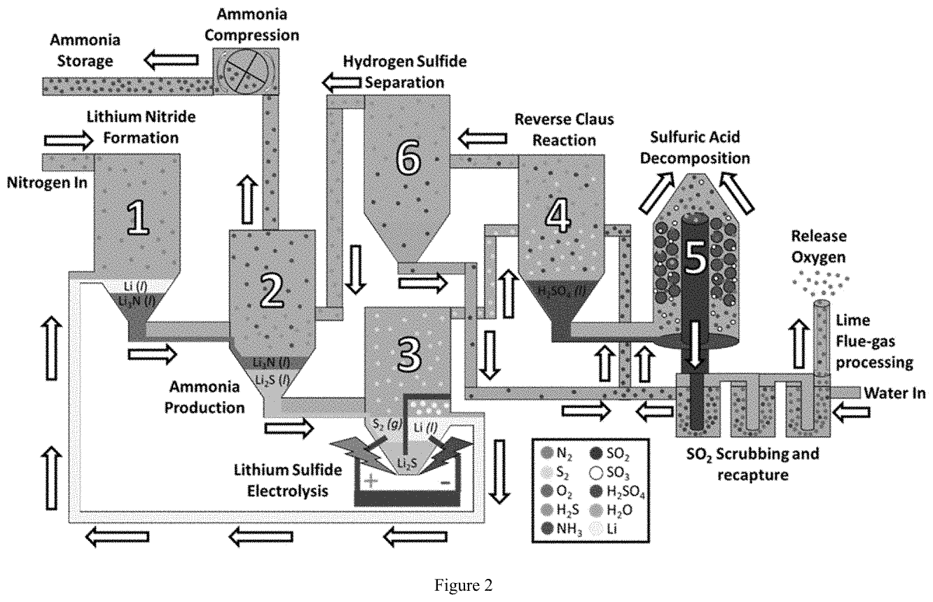

FIG. 1 shows the herein disclosed cycle next to the cycle proposed by McEnaney for comparison. A possible industrial process working off this proposed cycle could be run as follows, also detailed pictorially by a reaction diagram as shown in FIG. 2.

In steps 1-3, nitrogen gas will be sequestered from the air using pressure-swing absorption. This nitrogen will then be introduced into a crucible with molten lithium (mp: 180.5° C.) to form Li₃N. As the solution is stirred, the nitride will sink to the bottom of the molten metal where it can be collected (Density Li (1): 0.534 g/cm³, Li₃N (s): 1.27 g/cm³). The solid or liquid Li₃N collected from the first crucible (mp Li₃N: 813° C., bp Li: 1330° C.), will then be moved to a second crucible where it is exposed to H₂S gas, rapidly releasing NH₃ (g) and forming Li₂S (s).

While this reaction is more favorable at low temperatures (−984.2 kJ vs. −571.6 kJ at 900 K and 300 K respectively), it should still proceed at the melting point of Li₃N. The NH₃ (g) and H₂S (g) may be separated by densities in an industrial distillation column (Density NH₃: 0.73 kg/m³, H₂S: 1.36 kg/m³), with the heavier H₂S staying closer to the reaction, or using a membrane separation, with only the smaller NH₃ allowed out (size H₂S ˜267 pm, NH₃ ˜203 pm).

If this reaction is done at the melting point of Li₃N, the Li₂S may be separated from the continuously added Li₃N by density (density Li₂S: 1.66 g/cm³, Li₂N: 1.27), as the solid Li₂S will sink, continuously exposing fresh Li₃N to the H₂S (mp Li₃N: 813° C., Li₂S: 928° C.). The solid or liquid Li₂S may then be added to a third crucible either to be dissolved in a LiF eutectic at 800° C. or melted at 928° C. or above in a magnesium oxide crucible for the electrowinning of Li (1) and S₂ (g). During electrolysis, the sulfur will boil (Sulfur mp: 115.21° C., bp: 444.6° C.) while the Li (1) may be recaptured (as is done industrially in LiCl melts) by floating to the surface to be skimmed off the top. The molten Li may then be re-fed back into the first crucible to be reacted with N₂ (g), completing the first cycle.

In steps 4-7, gaseous sulfur produced by electrolysis of Li₂S may be injected into a crucible where it is mixed with a stoichiometric amount of steam at 300° C. or higher to produce hydrogen sulfide and sulfur dioxide (biproduct is SO₃). As the gasses rise higher in the column and cool, SO₂ will begin to form H₂SO₄, which will condense and pool at the bottom of the chamber. Similarly, liquid water will condense higher in the column, but as it falls it will be heated back into steam as it reaches the bottom of the chamber held at 300° C.

The steam, H₂S, SO₂, and gaseous sulfur (if any) may then be separated by condensation after extraction from the top of the distillation column. A compressor may increase the temperature required to separate H₂S from SO₂, so the system doesn't need to be cooled below RT. The entire system may be operated at higher pressures driven by the expansion of steam.

As the condensed sulfuric acid is removed from the bottom of the chamber, it may be necessary to allow the solution time to react with dissolved water and sulfur to form H₂S and SO₂ before the remaining H₂SO₄ is further heated. As the acid is heated above its boiling point it will begin to decompose into SO₃ and H₂O. These highly reactive gasses will then be sent through a SiC bayonet reactor and passed over 1 wt % Pt/Al₂O₃ catalysts to decompose the SO₃ in to SO₂ and O₂. These three gasses may be separated by passing through water, where differences in solubilities (at 40° C. O₂=˜0.03 g/L, SO₂=˜50 g/L H₂O, (˜1700× more soluble) will separate them. This water may then be further heated to steam and injected back into the reaction crucible to complete the cycle.

In sum, this example describes a newly disclosed cycle that further improves on the Bachman cycle, by using the reverse Claus process to regenerate needed hydrogen sulfide from elemental sulfur extracted from the lithium sulfide product of the ammonia production step. This disclosed cycle generates no carbon dioxide and can operate at atmospheric pressure. Because simple turbines may be used to convert thermal energy to electrical energy at between 20 and 35% efficiency, thermochemical cycles such as the disclosed cycle can more efficiently utilize the energy needed to drive the process.

The invention is not limited to the embodiments set forth in this disclosure for illustration but includes everything that is within the scope of the appended claims.

1. A method for making ammonia comprising:
(a) reacting lithium nitride with hydrogen sulfide to produce lithium sulfide and ammonia;
(b) reacting lithium with nitrogen to produce the lithium nitride that is used as a reactant in step (a);
(c) extracting lithium from the lithium sulfide that is produced in step (a) to produce the lithium that is used as a reactant in step (b), whereby a sulfur byproduct is produced; and
(d) reacting the sulfur byproduct of step (c) with a feedstock comprising water, hydrogen, a carbon-containing composition, or both water and a carbon-containing composition to produce the hydrogen sulfide that is used as a reactant in step (a).

2. The method of claim 1, wherein steps (a)-(d) occur:
(ii) simultaneously;
(ii) within one or more interconnected reaction chambers; or
(iii) in a multi-loop chemical cycle.

3.-8. (canceled)

9. The method of claim 2, wherein the feedstock used as a reactant in step (d) is added to the interconnected reaction chambers or to the multi-loop chemical cycle from a source that is external to the interconnected reaction chambers or to the chemical cycle.

10.-11. (canceled)

12. The method of claim 2, wherein the ammonia produced as a product in step (a) is removed from the interconnected reaction chambers or from the multi-loop chemical cycle.

13.-14. (canceled)

15. The method of claim 1, wherein one or more of steps (a)-(d) occur at less than 2.0 atmospheres of pressure, at less than 1.5 atmospheres of pressure, or at atmospheric pressure.

16.-17. (canceled)

18. The method of claim 1, wherein the feedstock comprises water, but does not comprise a carbon-containing composition.

19. The method of claim 18, wherein the method does not generate carbon dioxide.

20. The method of claim 18, wherein step (d) is performed using the Reverse Claus Process.

21. (canceled)

22. The method of claim 20, wherein step (d) produces a sulfuric acid byproduct.

23.-26. (canceled)

27. The method of claim 20, wherein step (d) directly or indirectly produces one or more undesirable byproducts selected from the group consisting of water, sulfur dioxide, sulfur trioxide, and other heavy gases.

28. The method of claim 27, wherein step (d) further comprises separating the hydrogen sulfide from the one or more undesirable byproducts.

29. (canceled)

30. The method of claim 18, wherein step (d) directly or indirectly produces oxygen as a byproduct.

31. (canceled)

32. The method of claim 1, wherein the feedstock comprises water and a carbon-containing composition.

33. The method of claim 32, wherein the carbon-containing composition comprises one or more components selected from the group consisting of biomass, a hydrocarbon, a plastic, and an oxide of carbon.

34.-40. (canceled)

41. The method of claim 32, wherein the mole ratio of C:O in the water and carbon-containing composition together is from 0.8:2 to 1.2:2, from 0.9:2 to 1.1:2, or from 0.95:2 to 1.05:2.

42.-45. (canceled)

46. The method of claim 32, wherein step (d) produces a carbon dioxide byproduct.

47. The method of claim 46, further comprising reacting the carbon dioxide byproduct with the ammonia produced in step (a) to produce urea.

48.-50. (canceled)

51. The method of claim 46, wherein performing step (d) further comprises removing the carbon dioxide byproduct.

52. The method of claim 51, wherein the removed carbon dioxide byproduct is frozen, is sequestered, or is reacted with ammonia to produce urea.

53.-55. (canceled)

56. The method of claim 1, wherein steps (a)-(c) occur within a flux of molten lithium-halogen salt.
